(19) United States
(12) Patent Application Publication
(10) Pub. No.: US 2008/0187998 A1
(43) Pub. Date: Aug. 7, 2008

(54) Sulfonamide-based oligomers and polymers for destabilization of biological membranes

(75) Inventors: Han Chang Kang, Salt Lake City, UT (US); You Han Bae, Salt Lake City, UT (US)

Correspondence Address: ALAN J. HOWARTH, P.O. BOX 1909, SANDY, UT, 84091-1909, US

(73) Assignee: UNIVERSITY OF UTAH RESEARCH FOUNDATION

(21) Appl. No.: 11/701,975

(22) Filed: Feb. 1, 2007

(51) Int. Cl. C12N 15/85; C07C 311/01; C12N 15/88; C12N 5/06; C12N 5/10

(52) U.S. Cl. 435/458; 435/320.1; 435/375; 564/80

(57) ABSTRACT

Oligomeric sulfonamides for use as endosomolytic reagents for transfection with polymeric or lipid-based vectors are described. A mixture of an oligomeric sulfonamide with a polymeric or lipid-based gene carrier and a nucleic acid results in a polyplex that exhibits 6-12-fold better gene expression than controls. A method of transfecting cells in vitro is carried out by contacting cultured mammalian cells with an effective amount of a polyplex.

### STATEMENT REGARDING FEDERALLY SPONSORED RESEARCH OR DEVELOPMENT

This invention was made with government support under grant no. DK 56884 from the National Institutes of Health. The government has certain rights in the invention.

### CROSS-REFERENCE TO RELATED APPLICATIONS

Not applicable.

### BACKGROUND OF THE INVENTION

This invention relates to gene delivery. More particularly, this invention relates to endosome-disrupting oligomers from sulfonamide derivatives for use in polymeric gene delivery.

Polymeric gene carriers are a potent alternative to viral vectors, which raise intrinsic safety concerns such as immunogenicity. However, polymeric vectors still suffer from low transfection rates in animal models and clinical applications despite having shown improved gene expression in cultured cells. Among several considerable strategies for effective polymeric transfection, the introduction of endosomolytic function is an important method. H. C. Kang, M. Lee & Y. H. Bae, Polymeric gene carriers, 15 Crit. Rev. Eukarot. Gene Expr. 317-342 (2005). This function endows facilitated endosomal escape of polyplexes, leading to preventing mass loss of plasmid DNA by enzymatic degradation in lysosomal compartments. Id.; Y. W. Cho et al., Polycation gene delivery systems: escape from endosomes to cytosol, 55 J. Pharm. Pharmacol. 721-734 (2003). Endosomal disruption is performed by fusogenic or endosomolytic groups that can be chemically or physically introduced into polyplexes. Currently, fusogenic peptides (i.e., KALA, GALA) and polycations with protonatable amine groups (i.e., polyethyleneimine (“PEI”), poly(L-histidine), poly(amidoamine), and their copolymers) have elicited endosomal breakage. Also, endosome-disrupting polyanions, such as poly(2-alkylacrylic acid) (“PAA”), have been applied for effective non-viral gene delivery. C. Y. Cheung et al., A pH-sensitive polymer that enhances cationic lipid-mediated gene transfer, 12 Bioconjug. Chem. 906-910 (2001); T. R. Kyriakides et al., pH-sensitive polymers that enhance intracellular drug delivery in vivo, 78 J. Control. Release 295-303 (2002); T. Kiang et al., Formulation of chitosan-DNA nanoparticles with poly(propyl acrylic acid) enhances gene expression, 15 J. Biomater. Sci. Polym. 1405-1421 (2004).

Endosomolytic activity of PAA was assessed by its hemolytic activity in the endosomal pH ranges. Depending on alkyl groups, poly(2-propylacrylic acid) (“PPAA”), poly(2-ethylacrylic acid) (“PEAA”), and poly(2-methylacrylic acid) (“PMAA”) showed different effective pH ranges of the hemolytic activity and induced different transfection efficiencies in cultured cells. N. Murthy et al., The design and synthesis of polymers for eukaryotic membrane disruption, 61 J. Control. Release 137-143 (1999); R. A. Jones et al., Poly(2-alkylacrylic acid) polymers delivery molecules to the cytosol by pH-sensitive disruption of endosomal vesicles, 372 Biochem. J. 65-75 (2003); C. Kusonwiriyawong et al., Evaluation of pH-dependent membrane-disruptive properties of poly(acrylic acid) derived polymers, 56 Eur. J. Pharm. Biopharm. 237-246 (2003); M. A. Yessine et al., Characterization of the membrane-destabilizing properties of different pH-sensitive methacrylic acid copolymers, 1613 Biochim. Biophys. Acta 28-38 (2003). Among these polyanions, PPAA and PEAA showed improved transfection on various cell lines and caused the membrane rupture of erythrocytes at endosomal pHs. Their pH-sensitive hemolytic activities were strongly influenced by their dose and molecular weight. C. Kusonwiriyawong et al., supra. Higher molecular weight PAAs induced better hemolytic activity than lower molecular weight ones. Unlike PPAA and PEAA, PMAA showed negligible effects on hemolytic activity and transfection efficiency. However, the limited library of PAAs as endosomolytic polymers could limit its applications at a specific pH.

Bae's group reported pH sensitivity of sulfonamide polymers and oligomers, S. I. Kang & Y. H. Bae, pH-induced solubility transition of sulfonamide-based polymers, 80 J. Control. Relase 145-155 (2002); K. Na & Y. H. Bae, pH-sensitive polymers for drug delivery, in Polymeric Drug Delivery Systems 129-194 (G. S. Kwon, ed., Taylor & Francis Group, Boca Raton 2005); K. Na & Y. H. Bae, Self-assembled hydrogel nanoparticles responsive to tumor extracellular pH from pullulan derivative/sulfonamide conjugate: characterization, aggregation, and adriamycin release in vitro, 19 Pharm. Res. 681-688 (2002); S. Y. Park & Y. H. Bae, Novel pH-sensitive polymers containing sulfonamide groups, 20 Macromol. Rapid Commun. 269-273 (1999), and showed various biomedical and pharmaceutical applications such as anti-cancer carriers, K. Na & Y. H. Bae, 19 Pharm. Res. 681-688 (2002); K. Na, K. H. Lee & Y. H. Bae, pH-sensitivity and pH-dependent interior structural change of self-assembled hydrogel nanoparticles and pullulan acetate/oligosulfonamide conjugate, 97 J. Control. Release 513-525 (2004); K. Na, K. H. Lee & Y. H. Bae, Adriamycin loaded pullulan acetate/sulfonamide conjugate nanoparticles responding to tumor pH: pH-dependent cell interaction, internalization and cytotoxicity in vitro, 87 J. Control. Release 3-13 (2003); V. A. Sethuraman, K. Na & Y. H. Bae, pH-responsive sulfonamide/PEI system for tumor specific gene delivery: In vitro study, 7 Biomacromolecules 64-70 (2006), protein delivery formulations, S. I. Kang & Y. H. Bae, A sulfonamide based glucose-responsive hydrogel with covalently immobilized glucose oxidase and catalase, 86 J. Control. Release 115-121 (2003), protein separation, S. I. Kang & Y. H. Bae, pH-dependent elution profiles of selected proteins in HPLC having a stationary phase modified with pH-sensitive sulfonamide polymers, 15 J. Biomater. Sci. Polym. Ed. 879-894 (2004), and injectable drug delivery depots, W. S. Shim, J. S. Yoo, Y. H. Bae & D. S. Lee, Novel injectable pH and temperature sensitive block copolymer hydrogel, 6 Biomacromolecules 2930-2934 (2005). Sulfonamides and their oligomers or polymers have a broad range of pKₐ3-11, depending on side groups. W. O. Foye, Principles of Medicinal Chemistry, (3d ed., Lea & Febiger, Philadelphia 1989). Sulfonamide groups are protonated at a pH lower than the pKₐ and lose negative charges. This phenomenon causes very sharp solubility transition at pH ranges as small as 0.2 pH units. K. Na & Y. H. Bae, pH-sensitive polymers for drug delivery, in Polymeric Drug Delivery Systems 129-194 (G. S. Kwon, ed., Taylor & Francis Group, Boca Raton 2005).

In view of the foregoing, it will be appreciated that providing oligomeric or polymeric sulfonamides and methods of use in gene delivery would be a significant advancement in the art.

### BRIEF SUMMARY OF THE INVENTION

It is a feature of an illustrative embodiment of the present invention to provide oligomeric or polymeric sulfonamides for use in gene delivery.

It is a feature of another illustrative embodiment of the invention to provide polyplexes containing endosomolytic reagents for use in gene delivery.

It is a feature of still another illustrative embodiment of the invention to provide a method of transfecting mammalian cells in vitro using polyplexes containing endosomolytic reagents.

These and other features can be addressed by an illustrative embodiment of the invention comprising an oligomeric sulfonamide represented by the formula:

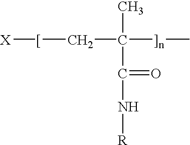

wherein X is (CH₃)₃—C— or H₂N—CH₂—CH₂—S—; R is a sulfonamide or N-substituted sulfonamide having a pKₐ of about 3 to 10; and n is about 2 to 300. Typically, the sulfonamide or N-substituted sulfonamide has a pKₐ of about 4 to 7.4. Illustrative sulfonamides or N-substituted sulfonamides include sulfamethizole, sulfadimethoxine, sulfadiazine, and sulfamerazine.

Another illustrative embodiment of the invention comprises a polyplex comprising a mixture of a nucleic acid, a polymeric or lipid-based gene carrier, and an oligomeric sulfonamide represented by the formula:

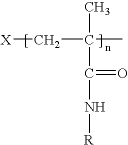

wherein X is (CH₃)₃—C— or H₂N—CH₂—CH₂—S—; R is a sulfonamide or N-substituted sulfonamide having a pKₐ of about 3 to 10; and n is about 2 to 300. Typically, the sulfonamide or N-substituted sulfonamide has a pKₐ of about 4 to 7.4. Illustrative sulfonamides or N-substituted sulfonamides include sulfamethizole, sulfadimethoxine, sulfadiazine, and sulfamerazine. Illustrative polymeric gene carriers are polycations, such as poly(L-lysine), polyethyleneimine, polyamidoamine, polyallylamine, polyornithine, copolymers or derivatives thereof, and mixtures thereof. Illustrative lipid-based gene carriers include liposomes.

Still another illustrative embodiment of the invention comprises a method for transfecting mammalian cells in vitro, the method comprising:

(a) culturing the mammalian cells in a selected growth medium; and

(b) contacting the cultured mammalian cells with an effective amount of a polyplex comprising a mixture of a nucleic acid, a positively charged polymer or lipid-based gene carrier, and an oligomeric sulfonamide represented by the formula:

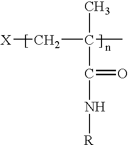

wherein X is (CH₃)₃—C— or H₂N—CH₂—CH₂—S—; R is a sulfonamide or N-substituted sulfonamide having a pKₐ of about 3 to 10; and n is about 2 to 300. Typically, the sulfonamide or N-substituted sulfonamide has a pKₐ of about 4 to 7.4. Illustrative sulfonamides or N-substituted sulfonamides include sulfamethizole, sulfadimethoxine, sulfadiazine, and sulfamerazine.

### BRIEF DESCRIPTION OF THE SEVERAL VIEWS OF THE DRAWINGS

FIG. 1 shows an illustrative scheme for synthesis of oligomeric sulfonamides according to the present invention.

FIGS. 2A-G shows the protons analyzed in the ¹H-NMR analysis summarized in Table 1. FIG. 2A shows the general structure of an oligomeric sulfonamide. FIGS. 2B-C show, respectively, the structures of X of FIG. 2A where X is (CH₃)₃—C— and H₂N—CH₂—CH₂—S—. FIGS. 2D-G show, respectively, the structures of R of FIG. 2A where R is in OSMT, OSDM, OSDZ, and OSMZ. Lower case letters refer to the indicated protons.

FIG. 3A shows acid-base titration curves of oligomeric sulfonamides (▾, OSMT; ▪, OSDM; ⋄, OSDZ; ▴, OSMZ) and controls (◯, PPAA; , NaCl).

FIG. 3B shows pH-dependent solubility transition of oligomeric sulfonamides (▾, OSMT; ▪, OSDM; ⋄, OSDZ; ▴, OSMZ) and controls (◯, PPAA; , NaCl).

FIG. 4 shows hemolysis by PPAA (control) and oligomeric sulfonamides (OSMT, OSDM, OSDZ, and OSMZ) at selected pH levels. Data points represent means±SEM; n=3.

FIGS. 5A-B show cytotoxicity of oligomeric sulfonamides (, OSMT; ▾, OSDM; ▪, OSDZ; ⋄, OSMZ) and controls (▴, PPAA; ◯, PEI25 kDa; □, PLL) to HEK293 and HepG2 cells, respectively.

FIGS. 6A-B show gel retardation assays of oligomeric sulfonamide-polyplexes and PPAA-polyplexes.

FIGS. 7A-B show zeta potentials and particles sizes, respectively, of oligomeric sulfonamide-polyplexes and PPAA-polyplexes. Controls were PLL/DNA complexes with charge ratios of 3. Data points represent means±SD.

FIGS. 8A-B show in vitro transfections assays of oligomeric sulfonamide-polyplexes and PPAA-polyplexes in HEK-293 cells and HepG2 cells, respectively. Transfection efficiency (RLU/mg protein) of PLL/DNA complexes (charge ratio=3) was set as unity. Data points represent means±SEM; n≧4.

FIG. 9 shows the effects of chloroquine and bafilomycin A₁ on transfection of OSDZ-polyplexes to HEK293 cells and HepG2 cells. Transfection efficiency (RLU/mg protein) of PLL/DNA complexes (charge ratio=3) was set as unity. Data points represent means±SEM; n≧4.

### DETAILED DESCRIPTION

Before the present oligomeric sulfonamides and methods are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise. Thus, for example, reference to a polyplex containing “a polymeric gene carrier” includes a mixture of two or more polymeric gene carriers, reference to “an oligomeric sulfonamide” includes reference to two or more of such oligomeric sulfonamides, and reference to “a nucleic acid” includes reference to a mixture of two or more nucleic acids.

Unless defined otherwise, all technical and scientific terms used herein have the same meanings as commonly understood by one of ordinary skill in the art to which this invention belongs.

In describing and claiming the present invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.” As used herein, “consisting of” and grammatical equivalents thereof exclude any element, step, or ingredient not specified in the claim. As used herein, “consisting essentially of” and grammatical equivalents thereof limit the scope of a claim to the specified materials or steps and those that do not materially affect the basic and novel characteristic or characteristics of the claimed invention.

As used herein, “polyplex” means a complex comprising a nucleic acid and a polymeric or lipid-based gene carrier. Illustrative polyplexes according to the present invention further comprise an oligomeric sulfonamide.

As used herein, “polymeric gene carrier” means a polymer useful for transfecting a cell with a nucleic acid. Typically, polymeric gene carriers are positively charged such that they form ionic bonds with negatively charged nucleic acids. Illustrative polymeric gene carriers include poly(L-lysine) (“PLL”), polyethyleneimine (“PEI”), poly(L-histidine), polyamidoamine, polyallylamine, polyornithine, and the like, and copolymers and derivatives thereof, and mixtures thereof.

As used herein, “lipid-based gene carrier” means lipid-containing compounds useful for transfecting a cell with a nucleic acid. Illustrative lipid-based gene carriers comprise liposomes.

As used herein, “nucleic acid” means single-stranded or double-stranded DNA, RNA, or DNA/RNA complexes. Illustrative nucleic acids include plasmids, small interfering RNAs (siRNAs), oligonucleotides, and the like, and mixtures thereof.

As used herein, “charge ratio” means a ratio of positive to negative charges in a polyplex. Polyplexes according to the present invention typically have a charge ratio of about 0.0001 to 10,000.

As used herein, “effective amount” means an amount of a polyplex that is nontoxic but sufficient to provide a selected effect and performance. For example, an effective amount of a selected polyplex is an amount sufficient to effect in vitro transfection of mammalian cells. Such effective amount can be determined by a person of ordinary skill in the art according to methods well known in the art without undue experimentation.

As used herein, “SDM” means sulfadimethoxine, “OSDM” means oligomeric sulfadimethoxine, “SDZ” means sulfadiazine, “OSDZ” means oligomeric sulfadiazine, “SMZ” means sulfamerazine, “OSMZ” means oligomeric sulfamerazine, “SMT” means sulfamethizole, and “OSMT” means oligomeric sulfamethizole.

As used herein, “oligomer” means a polymer of any length. Thus, the term “oligomeric sulfonamide” and similar terms are used herein without any particular intended size limitation, unless a particular size is otherwise stated. Generally, oligomeric sulfonamides according to the present invention have molecular weights from about 700 to about 100,000, which correspond to a range of about 2 to about 300 monomeric units. Typically, the oligomeric sulfonamides according to the present invention have molecular weights from about 1000 to about 33,000, which correspond to a range of about 3 to about 100 monomeric units. More typically, the oligomeric sulfonamides according to the present invention have molecular weights from about 1000 to about 10,000, which correspond to a range of about 3 to about 30 monomeric units.

Researchers have struggled to achieve clinically effective transfection rates and safety records with polymeric gene vectors. One approach for enhancing polymeric transfection is the introduction of endosomolytic functionality into polymer/gene complexes (“polyplexes”). According to an illustrative embodiment of the present invention, four different oligomeric sulfonamides were developed for achieving effective endosomal release of polyplexes. In endosomal pH ranges, these oligomeric sulfonamides showed a proton-buffering effect and a sharp solubility transition while exhibiting negligible hemolytic activity. In vitro transfection studies of oligomeric-sulfonamide-containing polyplexes showed 6-12-fold better gene expression than controls. These findings support oligomeric sulfonamides as potent endosomolytic anions for enhancing polymeric gene transfection.

According to an illustrative embodiment of the present invention, four different sulfonamides having pKₐ values from early endosomal pH to late endosomal pH were selected; sulfamethizole (SMT, pKₐ=5.45), sulfadimethoxine (SDM, pKₐ=6.1), sulfadiazine (SDZ, pKₐ=6.4-6.5), and sulfamerazine (pKₐ=7.0). Selected sulfonamides were oligomerized and the applicability of their oligomers was investigated in polymeric gene transfection.

### EXAMPLES

The following materials were used in the examples set out below. SDM (99%), SDZ (100%), SMZ (99.9%), poly(L-lysine)-HBr (PLL; M_{w}(viscosity)=27,400 Da, M_{w}(MALLS)=30,200 Da), branched poly(ethyleneimine) (PEI; M_{w}=25 kDa; “PEI25 kDa”), propylacrylic acid (99%), bafilomycin A₁ (91.3%), chloroquine diphosphate salt, 2-aminoethanethiol (98%), Dulbecco's phosphate buffered saline (“DPBS”), Dulbecco's modified Eagle's medium (“DMEM”), trypsin-EDTA, 2,2′,-azobis(2-methylpropionitrile) (“AIBN”), 3-(4,5-di-methylthiazol-2-yl)-2,5-diphenyltetrazolium bromide (“MTT”), and dimethylsulfoxide (“DMSO”) were purchased from Sigma-Aldrich Chemical Inc. (St. Louis, Mo.). SMT (98%) and fetal bovine serum (“FBS”) were bought from TCI (Tokyo, Japan) and GIBCO BRL (Grand Island, N.Y.), respectively. AIBN was purified by recrystallization twice in methanol and DMSO was distilled at 75° C./12 mmHg prior to use.

### Example 1

### Synthesis of Oligomeric Sulfonamides

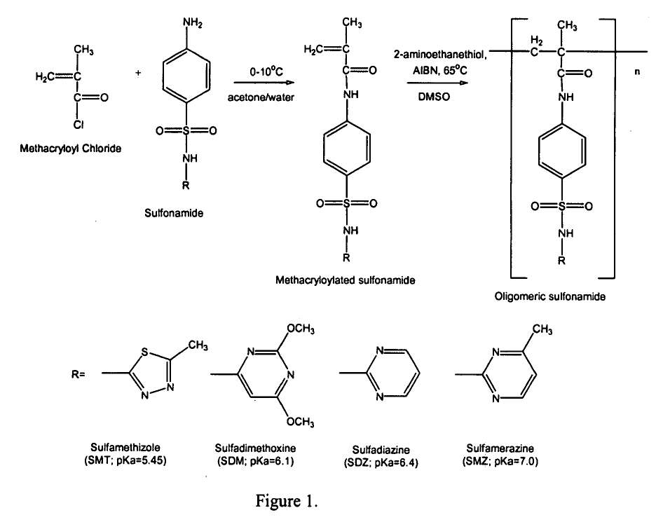

Methacryloylated sulfonamides were prepared as described in S. I. Kang & Y. H. Bae, pH-induced solubility transition of sulfonamide-based polymers, 80 J. Control. Relase 145-155 (2002); S. Y. Park & Y. H. Bae, Novel pH-sensitive polymers containing sulfonamide groups, 20 Macromol. Rapid Commun. 269-273 (1999); S. K. Han, K. Na & Y. H. Bae, Sulfonamide based pH-sensitive polymeric micelles: physicochemical characteristics and pH-dependent aggregation, 214 Colloid Surface A. Physicochem. Eng. Aspects 49-59 (2003). As shown schematically in FIG. 1, a sulfonamide (10 mmol) and NaOH (10 mmol) were dissolved in 40 mL of water-acetone mixture (water:acetone=1:1 (v/v)). Then, methacryloyl chloride (10 mmol) was slowly added into the sulfonamide solution in an ice-water bath (0-5° C.) with vigorous stirring. A condensation reaction between the methacryloyl chloride and the sulfonamide was carried out for 1 hr. NaOH acted as a scavenger of HCl generated during the condensation reaction. The precipitated product (methacryloylated sulfonamide) was filtered and dried in vacuo for at least 2 d at room temperature (RT). The methacryloylated sulfonamide was purified by recrystallization in methanol. Synthesis was verified by ¹H-NMR spectrophotometry (Varian Mercury 400).

Oligomeric sulfonamides were synthesized from methacryloylated sulfonamides by radical polymerization. A methacryloylated sulfonamide (1 mmol) was dissolved in 10 mL of distilled DMSO in the presence of AIBN (0.2 mol % of methacryloylated sulfonamide) as an initiator and 2-aminoethanethiol (0.4 mol % of methacryloylated sulfonamide) as a chain transfer agent. The reaction solution was bubbled with dry nitrogen gas for 30 min. The polymerization was carried out at 65° C. for 24 h. The product (oligomeric sulfonamide) was precipitated in excess deionized water. To remove unreacted monomers, the precipitated oligomer was dissolved in aqueous NaOH (pH 9) and dialyzed against water (adjusted to pH 9) with dialysis tubing (MWCO 2000) for 3 d. The final product was obtained after lyophilization. Characterization of the oligomer was measured by gel permeation chromatography (GPC; Agilent Technologies) for molecular weight and ¹H-NMR spectrophotometry for chemical structure.

Poly(propylacrylic acid) (“PPAA”), a well-known endosomolytic polyanion, was prepared from propylacrylic acid by a similar method.

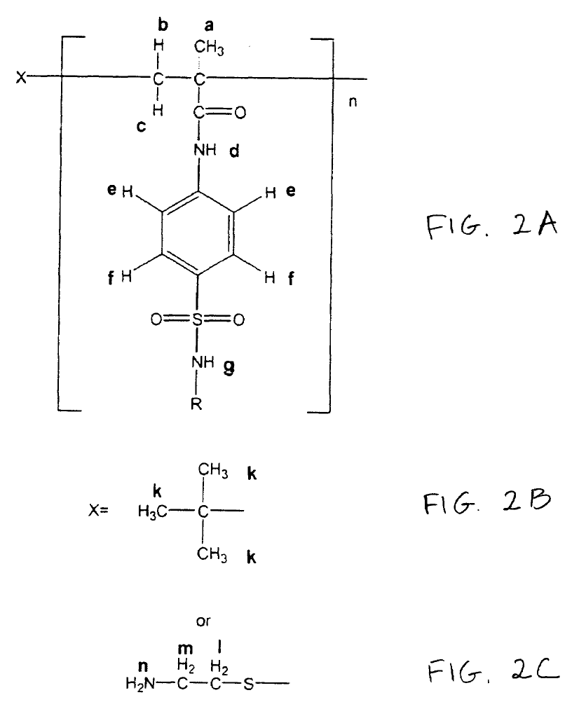

Syntheses of monomeric sulfonamides and oligomeric sulfonamides were confirmed by ¹H-NMR spectroscopy. S. K. Han, K. Na & Y. H. Bae, Sulfonamide based pH-sensitive polymeric micelles: physicochemical characteristics and pH-dependent aggregation, 214 Colloid. Surface A. Physicochem. Eng. Aspects 49-59 (2003); J. Turczan & T. Medwick, Identification of sulfonamides by NMR spectroscopy, 61 J. Pharm. Sci. 434-443 (1972). After conjugation between methacryloyl chloride and the sulfonamide, amine groups in the sulfonamide (8=5.9-6.1) had disappeared, whereas amide groups in the methacryloylated sulfonamide were formed and confirmed by a peak of CONH (δ=10.1˜10.2). Oligomeric sulfonamides synthesized from methacryloylated sulfonamides exhibited little change in chemical shifts (Δδ=0.01˜0.2) at protons (a-f, FIGS. 2A-G) toward lower ppm, probably because of the loss of a double bond from the methacryloylated sulfonamides. Peak analysis of oligomeric sulfonamides in ¹H-NMR spectra is summarized in Table 1.

TABLE 1

| δ value (ppm) | OSMT | OSDM | OSDZ | OSMZ |
|---|---|---|---|---|
| a | 1.92 | 1.93 | 1.93 | 1.93 |
| b | 5.52 | 5.51 | 5.50 | 5.49 |
| c | 5.80 | 5.79 | 5.79 | 5.79 |
| d | 9.84 | 9.88 | 9.82 | 9.81 |
| e | 7.69 | 7.64 | 7.67 | 7.70 |
| f | 7.61 | 7.64 | 7.59 | 7.59 |
| g | too broad | too broad | too broad | too broad |
| h | 2.33 | 3.60 | 8.05 | 7.89 |
| I | — | 5.51 | 6.32 | 6.21 |
| j | — | 1.93 | — | 2.06 |
| k | 1.22 | 1.23 | 1.22 | 1.12 |
| l | 2.66 | 2.65 | 2.65 | 2.65 |
| m | 2.30 | 2.30 | 2.30 | 2.30 |
| n | 1.55 | 1.50 | 1.55 | 1.50 |

Molecular weights and their distributions of the oligomeric sulfonamides were evaluated by GPC using DMSO and a low molecular weight PEG standard at 40° C. As shown in Table 2, the four oligomeric sulfonamides showed a number-average molecular weight (Mₙ) and a weight-average molecular weight (M_{w}) of about 2-3 kDa (about 5 to 9 monomeric units).

TABLE 2

| | OSMT | OSDM | OSDZ | OSMZ |
|---|---|---|---|---|
| Mₙ | 2002 | 2488 | 2251 | 1824 |
| M_{w} | 2099 | 2550 | 2318 | 1888 |

### Example 2

### Acid-Base Titration and Solubility Transition

Basic oligomeric sulfonamide solutions and PPAA solution were titrated with 0.1 N HCl for determination of proton buffering capacity and solubility transition against pH. Oligomeric sulfonamides (10 mg) prepared according to the procedure of Example 1 were dissolved in 150 mM NaCl aqueous solution (10 mL) with 100 μL of 1 N NaOH. A 3-mL aliquot was titrated with 0.1 N HCl at RT. Control solutions of PPAA and NaCl were similarly titrated. The changes of pH and transmittance (%) were monitored. Transmittance (%) was evaluated at 500 nm by a multi-modal microplate reader (SpectraMax M2; Molecular Devices, Sunnyvale, Calif.).

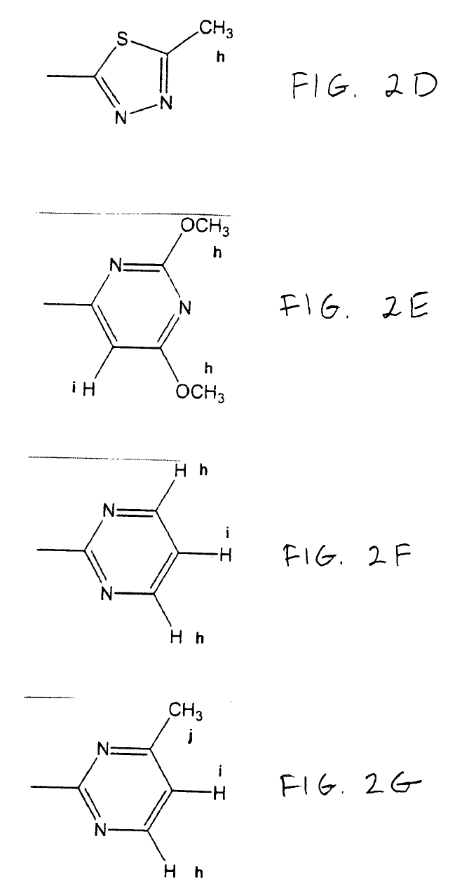

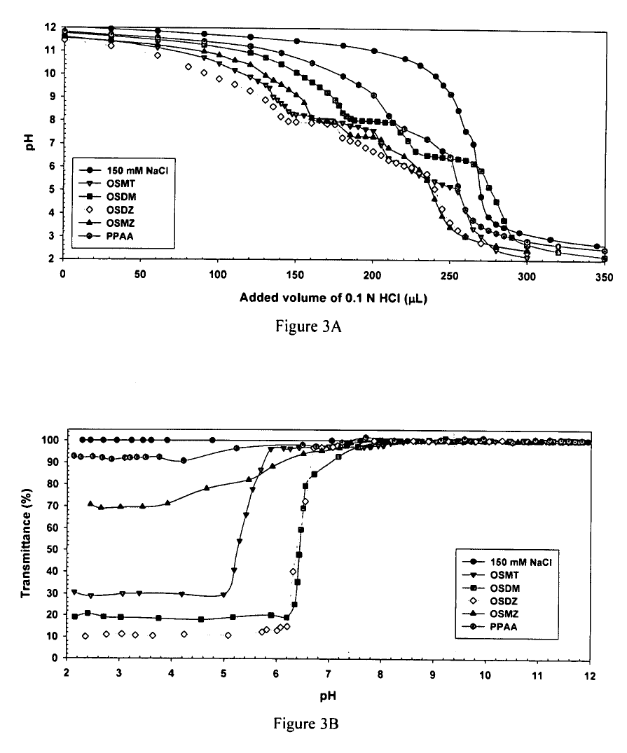

The proton-buffering capacity and pH-dependent solubility of transition of oligomeric sulfonamides was monitored by pH-titration and % transmittance measurements, respectively. As shown in FIG. 3A, addition of acid to oligomeric sulfonamide solutions (basic pH) showed buffering effect around pH 8, most probably due to one amine group at the end of an oligomeric sulfonamide originating from a chain transfer agent—aminoethanediol—used in synthesis. However, the major proton-buffering pH below pH 74. for oligomeric sulfonamides differ depending on their chemical structures. OSMT and OSDZ represented broad and weak pH buffering in pH ranges of 5.0-6.4 and 5.7-7.3, respectively, whereas OSDM and OSMZ induced short and strong pH buffering around pH 6.5 and 7.3, respectively. PPAA also showed short and weak pH buffering in the pH range 6.5-7.7. Apparent pKₐ values of oligomeric sulfonamides were about 0.1-0.3 of a pH unit higher than pKₐ values of sulfonamides, and this trend is consistent with a previous study using other polymeric sulfonamides. In addition, oligomeric sulfonamide solutions showed different solubility transitions (FIG. 3B). Oligomeric sulfonamides except OSMZ had relatively sharp solubility transitions that started around pH 7.4 and pH 7.6, respectively, and ended around pH 4.0. When compared to transmittance (100%) at pH 11.0, transmittance changes (ΔT %) of OSMZ and PPAA in their transition range were about 30% and 8%, respectively; whereas the ΔT % of OSMT and OSDZ were about 65% in pH 5.0-5.8 and 90% in pH 6.2-6.6, respectively. Interestingly, in the case of OSDM, two solubility transitions were observed: 20% reduction in a pH range of 6.4-7.8 and further additional reduction of 60% in a shore range of pH 6.2-6.4. It seems that these results might be strongly influenced by pH-dependent hydrophobicity of oligomeric sulfonamides.

### Example 3

### Hemolytic Activity

The hemolytic activity of oligomeric sulfonamides was investigated in pH-dependent tests. One SD rat was killed with isoflurane, and blood was obtained by cardiac puncture. Blood, collected in EDTA-containing tubes, was centrifuged at 1500 g for 15 min. The pellet washed three times with cold DPBS pH 7.4 by centrifugation at 1500 g for 15 min at 4° C. and resuspended in the same buffer. An oligomeric sulfonamide solution was added to the erythrocyte solution (10⁷ cells/mL) at different pHs (pHs 4.5, 5.5, 6.5, and 7.4) and was incubated for 60 min at 37° C. in a shaking water bath. The final concentration of the oligomeric sulfonamide solution was 20 μg/mL. The release of hemoglobin was determined after centrifugation (1500 g for 10 min) by photometric analysis of the supernatant at 541 nm. Complete hemolysis was achieved using 2% Triton X-100, yielding the 100% control value, and 0% hemolysis was deemed the value measured with DPBS buffer-treated erythrocyte solution (control). Hemolysis (%) of oligomeric sulfonamides was calculated with the following equation.

$$Hemolysis ( \% ) = \frac{{Abs}_{Sample} - {Abs}_{Control}}{{Abs}_{TritonX - 100} - {Abs}_{Control}} \times 100$$

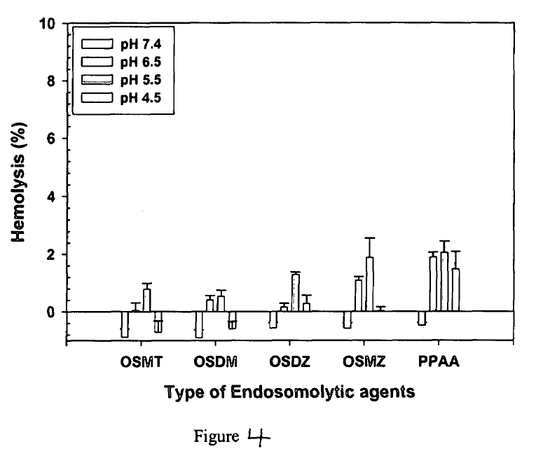

In pH-dependent hemolytic activity tests, oligomeric sulfonamides and PPAA (20 μg/mL/10⁷ erythrocytes) showed no hemolytic activity at pH 7.4 and also represented very low hemolytic activity (1-2%) at endosomal pHs (5.5-6.5) as shown in FIG. 4. These results deviated from expected values because oligomeric sulfonamides and PPAA showed proton-buffering and pH-dependent solubility transition, and the excellent hemolytic activity of PPAA has been reported. N. Murthy et al., supra; R. A. Jones et al., supra; C. Kusonwiriyawong et al., supra. These results suggest that the relatively low molecular weights of the oligomeric sulfonamides and PPAA may cause very low hemolysis. In addition, their hydrophobicities may not be sufficient to induce hemolysis, since it is expected that membrane rupture of erythrocytes is strongly affected by hydrophobicity of materials. R. A. Jones et al., supra.

### Example 4

### Cytotoxicity

Cytotoxicity of oligomeric sulfonamides and PPAA was assessed using 96-well plates and the MTT assay. HEK293 cells (human embryonic kidney cells) or HepG2 cells (human hepatocellular liver carcinoma cells) were seeded at a density of 2000 cells/well. The seeded cells were cultured for 24 hr prior to addition of oligomeric sulfonamides and PPAA. The cells were exposed to oligomeric sulfonamides and PPAA with various concentrations for 24 hr. Ten μL of MTT solution (5 mg/mL) was added to the cells (90 μL of culture medium), then the cells were incubated for an additional 4 h. After removing the MTT-containing medium, formazan crystals formed by living cells were dissolved in 0.1 mL of DMSO, and absorbance at 570 nm was evaluated using a SpectraMax® M2 microplate reader (Molecular Devices, Sunnyvale, Calif.). Cell viability (%) was calculated using the following equation:

$$Cell viability ( \% ) = \frac{{Abs}_{Sample} - {Abs}_{DMSO}}{{Abs}_{Control} - {Abs}_{DMSO}} \times 100$$

where Absₛₐₘₚₗₑ represents the absorbance from the cells treated with polyplexes, and Abs_{Control} represents the absorbance from the cells treated only with DPBS buffer. Abs_{DMSO} is the absorbance of DMSO.

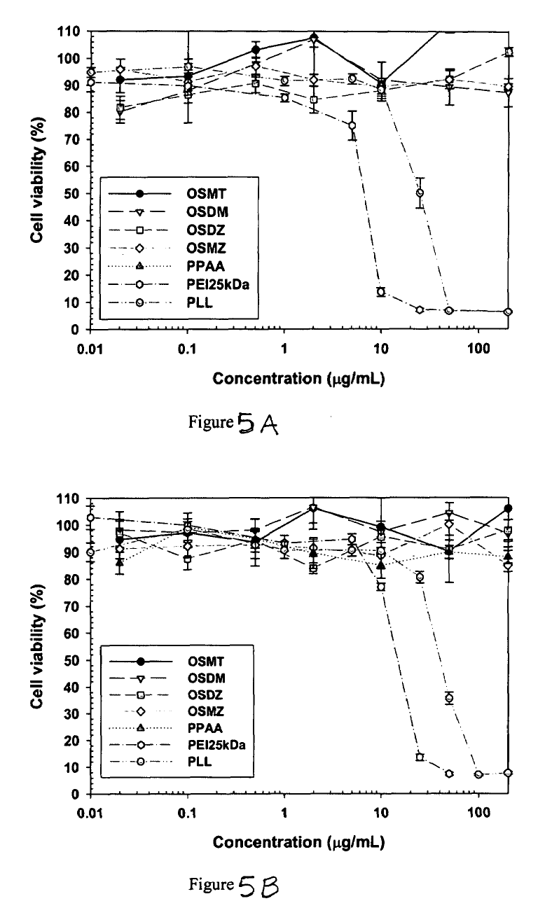

In these MTT-based cytotoxicity tests of oligomeric sulfonamides and PPAA to HEK293 and HepG2 cells (FIG. 5), different concentrations of oligomeric sulfonamides and PPAA (0 to 200 μg/mL/2000 cells) were applied. At their highest concentrations, viabilities were greater than 80% for cells exposed to both oligomeric sulfonamides and PPAA for 1 day. However, the IC₅₀ (material concentration that inhibits growth of 50% of cells relative to non-treated control cells) of PEI represented about 7 μg/mL for HEK293 cells and about 15 μg/mL for HepG2 cells. For PLL, IC₅₀'s were about 25 and 45 μg/mL for HEK293 and HepG2 cells, respectively. These results suggest that oligomeric sulfonamides are negligibly toxic.

### Example 5

### Preparation of Polyplexes

Polyplexes were prepared from a model plasmid DNA (pCMV-Luc, Promega, Madison, Wis.) and polymers. Ten μL of DNA solution (0.1 mg/mL; 1 μg of DNA) and an equal volume of PLL solution were mixed to make PLL/DNA complexes (20 μL). In the case of oligomeric-sulfonamide-containing (OSA-polyplexes) and PPAA-containing PLL/DNA complexes (PPAA-polyplexes), a total of 10 μL of DNA solution (1 μg of DNA) and oligomeric sulfonamide solution was mixed with an equal volume of PLL solution. The dose of PLL was dependent on the amount of oligomeric sulfonamide in the polyplexes. After incubation for 30 min at RT, the polyplexes were used in experiments. The charge ratio (+/−) of PLL/DNA complexes and oligomeric-sulfonamide-polyplexes and PPAA-polyplexes was 3.

### Example 6

### Gel Retardation Assay of Polyplexes

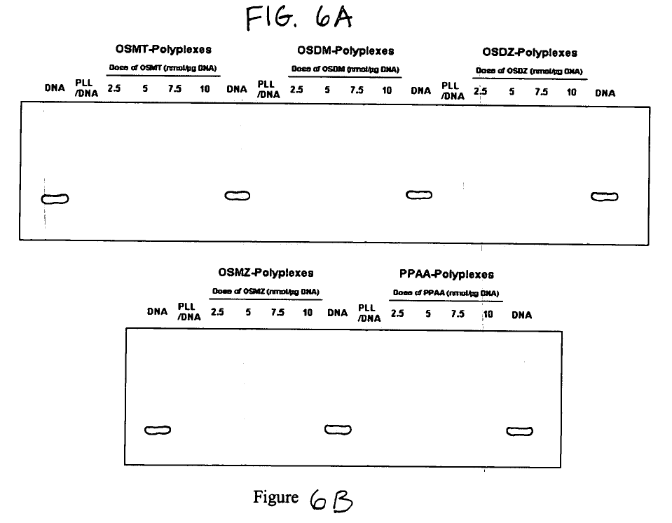

To determine potential their potential for gene delivery, the complexation of oligomeric-sulfonamide-containing PLL/DNA complexes (OSA-polyplexes) and PPAA-containing PLL/DNA complexes (PPAA-polyplexes) was monitored by agarose gel electrophoresis (FIGS. 6A-B). Polyplexes (10 μL; 0.5 μg DNA) were prepared according to the procedure of Example 5 except that polyplexes were prepared that contained 2.5, 5, 7.5, and 10 mmol/μg DNA. These polyplexes were loaded into a 0.8% agarose gel with ethidium bromide (0.5 μg/mL) and electrophoresed in TBE buffer at 100V for 60 min. The gel was viewed using a UV transilluminator (Gel Doc 2000 Gel Documentation System, Bio-Rad Laboratories, Hercules, Calif.).

Free DNA (control) migrated in the gel due to its negative charge characteristics. However, the migration of all OSA- and PPAA-polyplexes was retarded. These results suggest that OSA- and PPAA-polyplexes do not display negative charges on their surfaces.

### Example 7

### Surface Charge and Particle Size of Polyplexes

To check physicochemical characteristics of polyplexes, their particle size and surface charge were evaluated at 37° C. and pH 7.4. Polyplexes prepared according to the procedure of Example 5 were added to deionized water adjusted to pH 7.4 with 0.1 N NaOH. The concentration of DNA in the polyplexes was 1.5 μg/mL. Surface charge and particle size of polyplexes were measured using a Zetasizer 3000HS (Malvern Instrument, Inc, Worcestershire, UK) at a wavelength of 677 nm with a constant angle of 90° and 37° C.

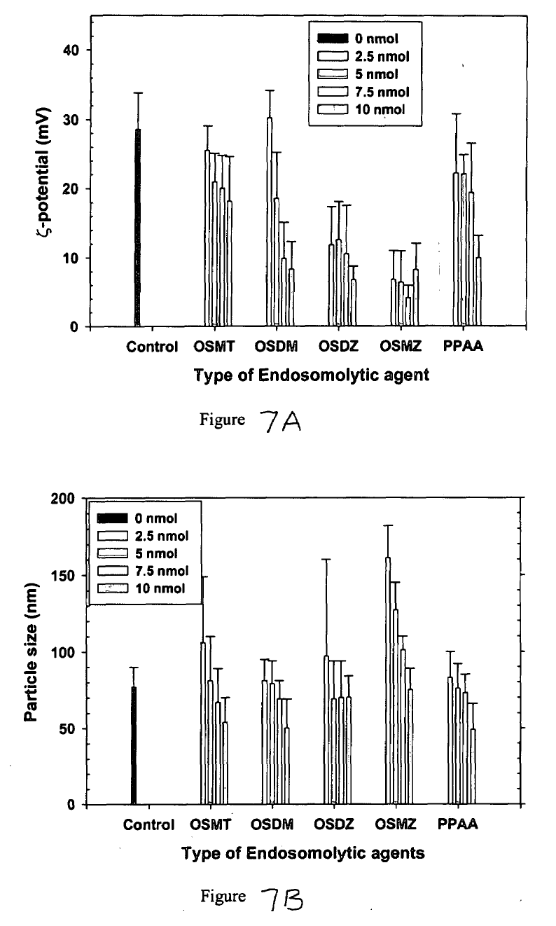

As shown in FIG. 7A, ∫-potential of PLL/DNA complexes (about 30 mV) was reduced by addition of oligomeric sulfonamides and PPAA, but the tested polyplexes had positively charged surfaces. These results support the results of the gel retardation studies (Example 6). Adding 5 nmol of oligomeric sulfonamides and PPAA formed surface charges of polyplexes with about 20 mV for OSMT-, OSDM-, and PPAA-polyplexes, about 12 mV for OSDZ-polyplexes, and about 6 mV for OSMZ-polyplexes. Also, the particle size of polyplexes decreased with increasing amounts of oligomeric sulfonamides and PPAA (FIG. 7B). Higher amounts of oligomeric sulfonamides and PPAA induced polyplexes with 50-70 nm hydrodynamic diameters, whereas lower amounts formed similar or larger particles compared to PLL/DNA complexes.

### Example 8

### In Vitro Transfection Study

The potential of oligomeric sulfonamides for non-viral gene delivery was investigated by in vitro transfection studies of PLL-based polyplexes to HEK293 cells and HepG2 cells. PPAA-polyplexes and PEI/DNA complexes were used as controls, because PPAA is a well-known endosomolytic polyanion and PEI is one of the best transfection agents. Additionally, polymeric transfection was studied in the presence of chloroquine (100 μM; a lysosomal disrupting agent) or bafilomycin A₁ (200 nM; a potent inhibitor of vacuolar H⁺-ATPase pumps) to determine whether OSA-polyplexes use endosomal vesicles.

In vitro transfection was performed in 6-well plates, and the cells were seeded at a density of 5×10⁵ cells/well for HepG2 cells and 1×100 cells/well for HEK293 cells. The seeded cells were cultured for 24 hr prior to adding polyplexes. One hour before transfection, the culture medium containing 10% serum (FBS) was replaced with serum-free DMEM. Polyplexes (20 mL; 1 μg of DNA) were prepared 30 min prior to transfection according to the procedure of Example 5. After dosing the polyplexes, the cells were transfected for 4 h, then, rinsed twice with Ca²⁺-free and Mg²⁺-free DPBS solution and incubated for 48 h with serum-containing DMEM. The cells were incubated in humidified air containing 5% CO₂ at 37° C. After transfection, the cells were rinsed twice with Ca²⁺-free and Mg²⁺-free DPBS solution and lysed using a reporter lysis buffer (200 mL/well). Relative luminescence units (RLU) were evaluated by the manufacturer's protocol for luciferase assay (Promega, Madison, Wis.). Protein content in the cells was evaluated by BCA™ protein assay.

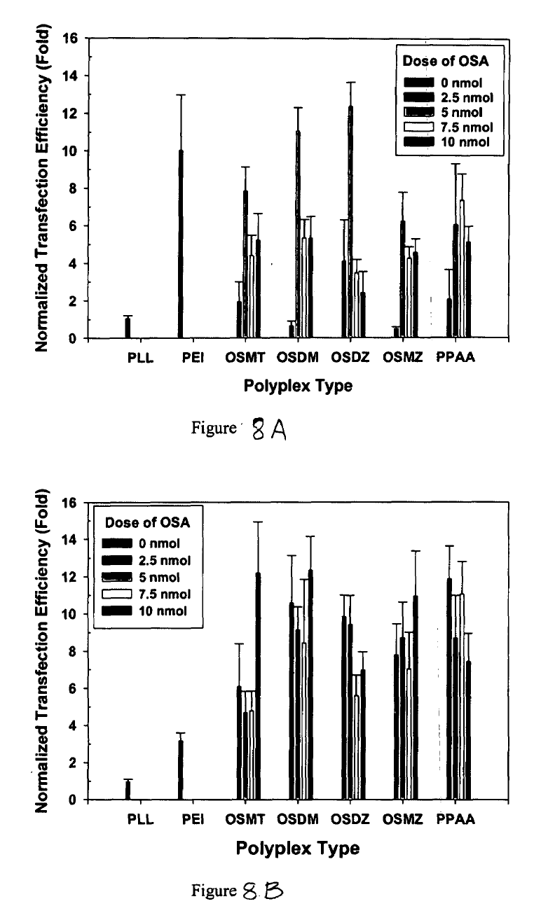

As shown in FIGS. 8A-B, the transfection results using OSA-polyplexes showed different levels of enhancement depending on the type of oligomeric sulfonamide, amount of oligomeric sulfonamide, and cell type. However, OSA- and PPAA-polyplexes caused 6-12-fold higher transfection efficiencies compared to the control PLL/DNA complexes.

For HEK293 cells, 5 nmol of oligomeric sulfonamide led to the highest transfection, but PPAA-polyplexes showed the best result at 7.5 nmol (FIG. 8A). OSDM- and OSDZ-polyplexes showed about 11-fold and 12-fold higher gene expression than PLL/DNA complexes, respectively, and were almost equivalent to PEI/DNA complexes (N/P=5). Also, these polyplexes were superior to PPAA-polyplexes. In addition, the effect of PLL dose was assessed, because higher oligomeric sulfonamide dose requires more PLL dose to form a fixed charge ratio (+/−=3). However, higher charge ratios of PLL/DNA complexes showed similar transfection rates to PLL/DNA complexes at a charge ratio of 3 (data not shown).

For HepG2 cells, OSA-polyplexes also showed transfection enhancement (FIG. 5B). OSA- and PPAA-polyplexes yielded 10-12-fold higher transfection rates than PLL/DNA complexes. However, higher gene expression of OSA-polyplexes at 7.5 and 10 nmol could have been influenced by the effect of PLL dose. PLL/DNA complexes in amounts used in OSA-polyplexes having 7.5 and 10 nmol oligomeric sulfonamides formed PLL/DNA complexes having charge ratios of 10.5 and 13, respectively, in the absence of oligomeric sulfonamides. Under these conditions, PLL/DNA complexes (+/−=10.5 or 13) yielded 3,4-fold and 2,3-fold better transfection, respectively, than PLL/DNA complexes (+/−=3). The transfection efficacy of OSA-polyplexes was superior to that of PEI/DNA complexes (N/P=5). However, although low N/P ratios of PEI/DNA complexes have been frequently used due to PEI cytotoxicity, high N/P ratios of PEI/DNA complexes exhibited better transfection efficiency using HepG2 cells. M. Morimoto et al., Molecular weight-dependent gene transfection activity of unmodified and galactosylated polyethyleneimine on hepatoma cells and mouse liver, 7 Mol. Ther. 254-261 (2003). Thus, OSA-polyplexes were compared with PEI/DNA complexes with high N/P ratios (N/P=10 and 15). These PEI/DNA complexes showed about 10-fold better gene expression than OSA-polyplexes (data not shown).

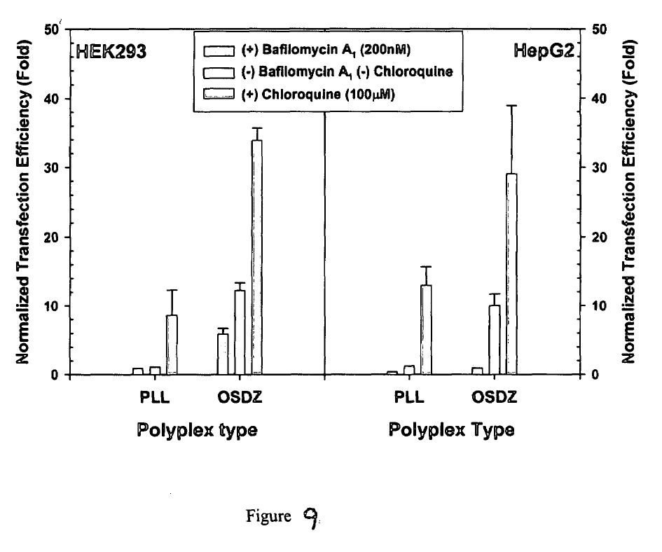

In addition, in vitro transfection studies in the presence of chloroquine (100 μM) or bafilomycin A₁ (200 nM) (FIG. 9) were carried out to assess whether the OSA-polyplexes use endosomal vesicles for intracellular trafficking. For HEK293 and HepG2 cells, chloroquine enhanced transfection of OSDZ-polyplexes and PLL/complexes, whereas bafilomycin A₁ reduced their transfection rates.

### CONCLUSION

Sulfonamides having pKₐ 's in the range of endosomal pHs were selected in certain illustrative embodiments of the present invention. Oligomeric sulfonamides were synthesized for effective endosomal disruption. Synthesized oligomeric sulfonamides demonstrated proton buffering capacity and solubility changes at endosomal pH. Oligomeric sulfonamide-polyplexes showed a positively-charged surface and formed particles about 50-150 nm in hydrodynamic diameter. Oligomeric sulfonamide-polyplexes improved polycation-based transfection, and the cytotoxicity of oligomeric sulfonamides against HEK293 and HepG2 cells was not significant. The effects of chloroquine and bafilomycin A₁ on the transfection of oligomeric sulfonamide-polyplexes suggest that oligomeric sulfonamides could influence the endosomal membrane during transfection. These findings support oligomeric sulfonamides as new endosomolytic reagents that improve transfection efficacy of plasmid DNA as well as siRNA and oligonucleotides in polymeric and lipid-based gene delivery.

The subject matter claimed is:

1. An oligomeric sulfonamide represented by the formula: 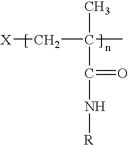
wherein X is (CH₃)₃—C— or H₂N—CH₂—CH_{2-S}—; R is a sulfonamide or N-substituted sulfonamide having a pKₐ of about 3 to 10; and n is about 2 to 300.

2. The oligomeric sulfonamide of claim 1 wherein R is sulfamethizole.

3. The oligomeric sulfonamide of claim 1 wherein R is sulfadimethoxine.

4. The oligomeric sulfonamide of claim 1 wherein R is sulfadiazine.

5. The oligomeric sulfonamide of claim 1 wherein R is sulfamerazine.

6. A polyplex comprising a mixture of a nucleic acid, a polymeric or lipid-based gene carrier, and an oligomeric sulfonamide represented by the formula: 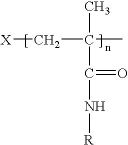
wherein X is (CH₃)₃—C— or H₂N—CH₂—CH₂—S—; R is a sulfonamide or N-substituted sulfonamide having a pKₐ of about 3 to 10; and n is about 2 to 300.

7. The polyplex of claim 6 wherein R is sulfamethizole.

8. The polyplex of claim 6 wherein R is sulfadimethoxine.

9. The polyplex of claim 6 wherein R is sulfadiazine.

10. The polyplex of claim 6 wherein R is sulfamerazine.

11. The polyplex of claim 6 wherein the nucleic acid comprises a plasmid, a small interfering RNA, or an oligonucleotide.

12. The polyplex of claim 6 wherein the polymeric or lipid-based gene carrier comprises a polycation or liposome.

13. The polyplex of claim 6 wherein the polyplex has a charge ratio of about 0.0001 to 10,000.

14. The polyplex of claim 13 wherein the polyplex has a charge ratio of about 3.

15. A method for transfecting mammalian cells in vitro, the method comprising:
(a) culturing the mammalian cells in a selected growth medium; and
(b) contacting the cultured mammalian cells with an effective amount of a polyplex comprising a mixture of a nucleic acid, a positively charged polymer or lipid-based gene carrier, and an oligomeric sulfonamide represented by the formula: 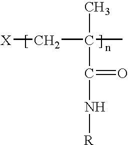
wherein X is (CH₃)₃—C— or H₂N—CH₂—CH₂—S—; R is a sulfonamide or N-substituted sulfonamide having a pKₐ of about 3 to 10; and n is about 2 to 300.

16. The method of claim 15 wherein R is sulfamethizole.

17. The method of claim 15 wherein R is sulfadimethoxine.

18. The method of claim 15 wherein R is sulfadiazine.

19. The method of claim 15 wherein R is sulfamerazine.

20. The method of claim 15 wherein the nucleic acid comprises a plasmid, a small interfering RNA, or an oligonucleotide.
